(19) United States
(12) Patent Application Publication
(10) Pub. No.: US 2013/0110068 A1
(43) Pub. Date: May 2, 2013

(54) Array Of Disposable Absorbent Articles

(71) Applicants: Denise Janet Nelson, Hortonville, WI (US); Rebecca Catherine Mohr, Appleton, WI (US); Kathryn Louise Stewart, Appleton, WI (US); Christopher Daniel McCarthy, Wexford, PA (US)

(72) Inventors: Denise Janet Nelson, Hortonville, WI (US); Rebecca Catherine Mohr, Appleton, WI (US); Kathryn Louise Stewart, Appleton, WI (US); Christopher Daniel McCarthy, Wexford, PA (US)

(21) Appl. No.: 13/285,118

(22) Filed: Oct. 31, 2011

(51) Int. Cl. A61F 13/49

(52) U.S. Cl. 604/385.01

(57) ABSTRACT

An array of disposable absorbent article configurations designed for daytime use by wearers at different levels of cooperation includes a first disposable diaper configuration including features configured to accommodate a first level of cooperation, wherein the first disposable diaper configuration is available in a first plurality of sizes; a second disposable diaper configuration including features configured to accommodate a second level of cooperation, the second level of cooperation being different from the first level of cooperation, wherein the second disposable diaper configuration is available in a second plurality of sizes; and a third disposable diaper configuration including features configured to accommodate a third level of cooperation, the third level of cooperation being different from the second level of cooperation, wherein the third disposable diaper configuration is structurally different from the second disposable diaper configuration, and wherein the third disposable diaper configuration is available in the second plurality of sizes.

### BACKGROUND

Disposable absorbent articles such as diapers are designed to absorb and contain bodily waste to prevent soiling of the body and clothing. Such disposable absorbent articles typically are available in a single design in different sizes to fit a variety of wearers ranging from newborns to active toddlers. The design of the diaper typically affects performance including the ability to absorb and contain bodily waste.

The problem with a one design fits all approach is that a single configuration might not be appropriate for every level of activity and capability. For instance, caregivers of newborns and immobile infants might desire a soft, yet bulky design that wraps the wearer and is gentle on the wearer's skin whereas once the infant becomes actively mobile it might be more desirable to have a contoured trim fit design providing the wearer more freedom of movement. Other designs might also be desired as the toddler grows and becomes more and more capable of independent activity, particularly during diaper changing time. For instance, a pull on diaper design might be beneficial for toddlers resisting the dressing experience. The level of cooperation demonstrated by a wearer with respect to a caregiver is an important determiner of the types of product features desired in a disposable absorbent article.

### SUMMARY

It is difficult to design a single product configuration exhibiting each of these attributes. Thus, there is a need for a variety of disposable diaper configurations matching a particular wearers capabilities, level of activity, and level of cooperation with a caregiver.

Most consumers think that a pull on pant configuration is for potty training or when the child is sturdy enough to hold weight on their feet. However, slip-on or pull-on diapers are another form of a diaper that consumers could use to more easily diaper an active baby (4+ months), even before they are walking and/or standing. This is a new behavior for consumers so it can be difficult for consumers to understand the concept without a great deal of explanation.

The present disclosure is generally directed to an array of disposable absorbent article configurations designed for daytime use by wearers at different levels of cooperation, the array including a first disposable diaper configuration including features configured to accommodate a first level of cooperation, wherein the first disposable diaper configuration is available in a first plurality of sizes; a second disposable diaper configuration including features configured to accommodate a second level of cooperation, the second level of cooperation being different from the first level of cooperation, wherein the second disposable diaper configuration is available in a second plurality of sizes; and a third disposable diaper configuration including features configured to accommodate a third level of cooperation, the third level of cooperation being different from the second level of cooperation, wherein the third disposable diaper configuration is structurally different from the second disposable diaper configuration, and wherein the third disposable diaper configuration is available in the second plurality of sizes.

The present disclosure is also generally directed to an array of disposable absorbent article configurations designed for daytime use by wearers at different levels of cooperation, the array including a first disposable diaper configuration including features configured to accommodate a first level of cooperation, wherein the first disposable diaper configuration is available in a first plurality of sizes; a second disposable diaper configuration including features configured to accommodate a second level of cooperation, the second level of cooperation being different from the first level of cooperation, wherein the second disposable diaper configuration is available in a second plurality of sizes, and wherein at least one of the second plurality of sizes is larger than each of the first plurality of sizes; and a third disposable diaper configuration including features configured to accommodate a third level of cooperation, the third level of cooperation being different from the second level of cooperation, wherein the third disposable diaper configuration is structurally different from the second disposable diaper configuration, and wherein the second and third disposable diaper configurations include parallel progressions of sizes.

The present disclosure is also generally directed to an array of disposable absorbent article configurations designed for daytime use by wearers at different levels of cooperation, the array including a first disposable diaper configuration including features configured to accommodate a first level of cooperation; a second disposable diaper configuration including features configured to accommodate a second level of cooperation, the second level of cooperation being different from the first level of cooperation, wherein the second disposable diaper configuration is available in a second plurality of sizes, and wherein the first and second disposable diaper configurations each have an ear and a fastening tab; and a third disposable diaper configuration including features configured to accommodate a third level of cooperation, the third level of cooperation being different from the second level of cooperation, wherein the third disposable diaper configuration is available in the second plurality of sizes, and wherein the third disposable diaper configuration includes a side panel and a pre-fastened, refastenable side seam.

The foregoing systems and devices can be employed relative to disposable diapers and other absorbent articles having a range of sizes and a variety of designs.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 is a schematic view of an example of a first disposable diaper configuration in the array of the present application;

FIG. 2 is a schematic view of an example of a second disposable diaper configuration in the array of the present application;

FIG. 3 is a schematic view of an example of a third disposable diaper configuration in the array of the present application;

FIG. 4 is a schematic view of an example of a fourth disposable article configuration in the array of the present application; and

FIG. 5 illustrates an example of a parallel sequence of sizes and disposable diaper configurations available to a consumer.

Repeat use of reference characters in the present specification and drawings is intended to represent the same or analogous features or elements of the present invention. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects of the present invention only, and is not intended as limiting the broader aspects of the present invention.

The present disclosure is directed to an array of disposable absorbent article configurations including parallel sequential paths of designs corresponding to a wearers levels of cooperation and levels of activity. For instance, the array can include a first disposable diaper configuration designed for newborns and immobile infants, where the wearer is not able to significantly resist an article change. The array can also include a second disposable diaper configuration for older but not quite mobile infants, where the wearer is not able to or generally does not significantly resist an article change. Further, the array can include a third disposable diaper configuration designed for more active babies and toddlers who might or might not be mobile, but who are capable of resisting an article change. Other disposable article configurations designed for those or subsequent levels of cooperation discussed below. Packaging and other merchandising elements for such articles can include indicia exhibiting pictorial representations of the disposable article configurations fitted to wearers at respective levels of cooperation enabling a consumer to identify the appropriate configuration that matches a particular wearer's level of cooperation. The system is particularly useful for merchandising multiple disposable article configurations that are available in overlapping size ranges along parallel paths.

As used herein, the term “absorbent article” refers to devices that are designed to absorb and contain bodily exudates, and, more specifically, refers to devices that are placed within, against, or in proximity to, the body of the wearer to absorb and contain the various exudates discharged from the body. As used herein, the term “level of cooperation” refers to level of emotional and cognitive maturity and/or the physical abilities of an individual including locomotion, mobility, motor skills, and coordination, particularly with respect to how cooperative a wearer is with a caregiver during an article change. As used herein, the term “diaper” refers to an absorbent article generally used by infants and incontinent persons that is worn about the lower torso of the wearer. As used herein, the term “chassis” refers to the main structure of the diaper with other features added to form the composite diaper structure. The term “disposable” is used herein to describe absorbent articles that are generally not intended to be laundered or otherwise restored or reused as an absorbent article (in other words, they are generally intended to be discarded after a single use). References to identifying a “size” are used herein to include a direct or indirect identification of a disposable diaper or other absorbent article size, such as by number or letter (for example, “Size 3” or “Size A”), by direct description (for example, “Small” or “Large”), or by any combination thereof. As used herein, “indicia” is an identifying marking that can include words and/or graphics describing a product in use. The products described herein are limited to those designed for general or daytime use for clarity of description and differentiation. Products designed with extra absorbency, for example, for nighttime use can be considered separately; a similar scheme can be applied to products specifically designed for night-time or overnight use.

The array of absorbent articles and merchandising system described herein can be applicable to a number of absorbent articles. For instance, adult incontinence garments can be made available in a variety of designs depending on a wearers level of cooperation. However, a preferred aspect includes an assortment of disposable diapers made available in a variety of product configurations. Each configuration includes distinguishable characteristics comprising structural differences addressing a wearers level of cooperation.

The levels of cooperation can range from newborns to active toddlers seeking independence. The levels of cooperation model focuses primarily on the time spent changing a diaper or other absorbent article, whereas traditional classifications tend to focus on activities during the time in which such an article is worn.

For instance, a first level of cooperation can cover a pre-locomotive phase and include newborns in a bonding stage with mom. At this first level, infants are generally uncoordinated, unable to support themselves, and have activity levels generally limited to eating, sleeping, and eliminating waste. As a result, the infant cannot generally assist in a diaper change, but is also unable to significantly resist a diaper change.

Second and third levels of cooperation can cover a pre-locomotive to locomotive phase of somewhat older babies or toddlers. At these levels, babies are more active; some can lift their heads, some can kick a leg over but not necessarily roll over, and some develop improved hand-eye coordination. With other children, the second and third levels can include sitting and mobility in the form of scooting, rolling, and crawling, and can also include toddlers whose level of activity includes standing, walking, and beginning to run. What distinguishes the second and third levels of cooperation is the amount of cooperation exhibited by the baby or toddler during an absorbent article change. In the second level of cooperation, the baby or toddler cannot generally assist in a diaper change, but is also unable to or simply does not significantly resist a diaper change. In the third level of cooperation, the baby or toddler also cannot generally assist in a diaper change, but is also able to significantly resist a diaper change by blocking changing efforts, or by being squirmy, mobile, or otherwise difficult to control.

A fourth level of cooperation can cover a locomotive, toilet training child who might be capable of assisting in a change, and can be changed whether the child is cooperative or resistant.

It should be noted that the levels of cooperation are not necessarily dictated by the age, size, or developmental level of the infant/baby/toddler, although those factors can influence the level of cooperation. Children develop and grow at different rates. For example, of two children of similar ages, sizes, and developmental levels, one might be in the second level of cooperation due to the ease with which that child can be changed, while the other might be in the third level of cooperation due to the difficulty encountered when changing that child.

It should also be noted that the levels of cooperation are not divided by strict rules or qualifications. The levels of cooperation provide guidance to a product developer and to a caregiver with respect to which of an array of disposable absorbent products might be better suited for a particular child.

Diapers are typically offered in a sequence of sizes that may have overlapping weight ranges, but that are designed to match the sequence of growth of a child. As the child grows, a series of articles with different sizes and structures can be needed to accommodate the child's size and the expected level of activity during product wear. As a result, a diaper with a given structure in a given size might be appropriate for one child but not for another if the levels of cooperation of those children during changes is different. The array of absorbent articles described herein is configured in parallel paths with at least two different types of diapers offered in a given size to accommodate different levels of cooperation. In this array system, the level of cooperation and the structure of the product are key to store shelf navigation and product selection.

The array of diapers described herein provides a series of diapers for each of the second and third levels of cooperation, wherein one series for the second level of cooperation more closely resembles a traditional diaper, and wherein the other series for the third level of cooperation is configured to be slipped on a child with the child in any position, including lying and standing. The two series are offered in parallel in the same or similar sizes to accommodate children of similar size but of different levels of cooperation. Both article structures have the same leakage protection, and the same or similar snug yet comfortable fit. The series of diapers configured to be slipped on a child further include sides that are easy to open for removal, repositioning, or donning without removing the child's clothing, such as described in co-pending U.S. patent application Ser. No. 12/956,258, filed on Nov. 30, 2010, which is incorporated herein by reference to the extent it does not conflict herewith. In one aspect of the present application, the absorbency of the second disposable diaper configuration at a given size is substantially the same as the absorbency of the third disposable diaper configuration at the given size.

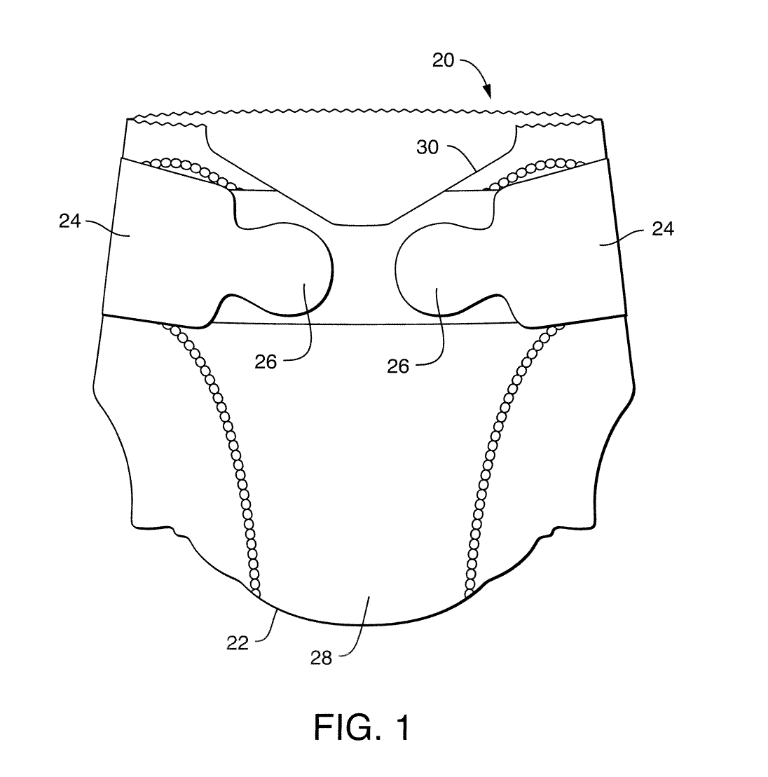

More specifically, the array of disposable article configurations described herein is designed to fit wearers at different levels of cooperation. The array includes a first disposable diaper configuration 20 intended for use in conjunction with the first level of cooperation. An example of a first disposable diaper configuration 20 is illustrated in FIG. 1. The first disposable diaper configuration 20 includes a chassis 22 designed with specific features to correspond to a wearer's pre-locomotive phase of development. The first disposable diaper configuration 20 has generally what is considered a typical or standard diaper configuration including a chassis 22, stretch or non-stretch ears 24, softer, gentler materials for tender skin, and fastening tabs 26 configured to allow a caregiver to close the diaper around the child. The first disposable diaper configuration 20 can also include a wetness indicator 28 to help a caregiver know when to change the diaper. In newborn sizes, the first disposable diaper configuration 20 can include an umbilical cord cutout 30.

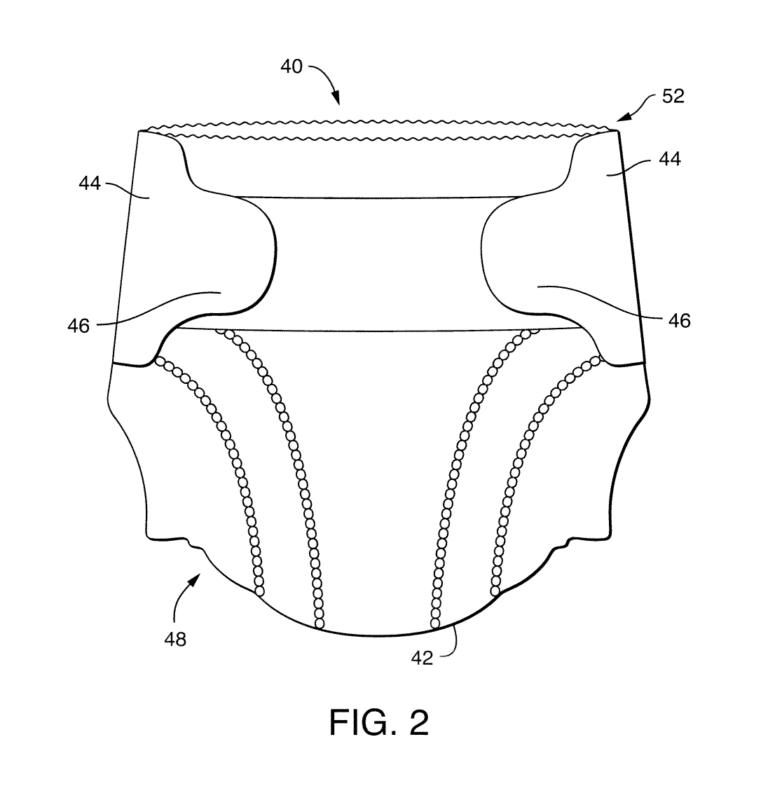

The array includes a second disposable diaper configuration 40 intended for use in conjunction with the second level of cooperation. An example of a second disposable diaper configuration 40 is illustrated in FIG. 2. The second disposable diaper configuration 40 includes a chassis 42 designed with specific features to correspond to a wearers more active phase of development. The second disposable diaper configuration 40 has generally what is considered a typical or standard diaper configuration including a chassis 42, stretch or non-stretch ears 44, higher absorbency, engaging graphics 48, a shaped pad (not shown) to accommodate active play, and more secure, stretch or non-stretch fastening tabs 46 configured to allow a caregiver to fasten the diaper back to front to accommodate changes with the child in a supine position.

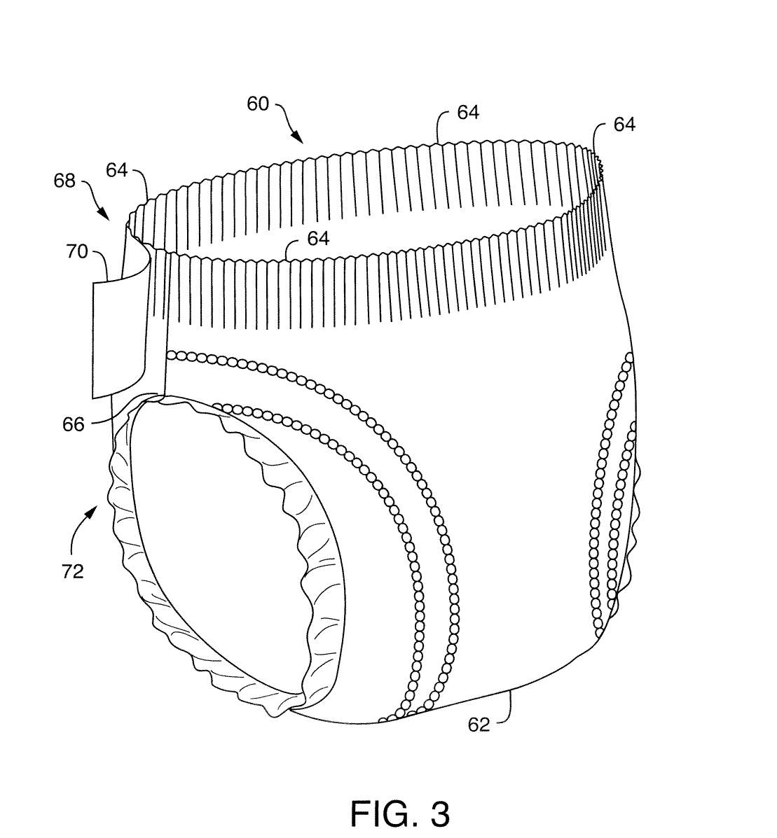

The array includes a third disposable diaper configuration 60 intended for use in conjunction with the third level of cooperation. An example of a third disposable diaper configuration 60 is illustrated in FIG. 3. The third disposable diaper configuration 60 includes a chassis 62 designed with specific features to correspond to a wearers increased ability to resist changes. The third disposable diaper configuration 60 has generally a pre-fastened configuration that allows a caregiver to pull the product onto a child. The third disposable diaper configuration 60 can also be unfastened by the caregiver and applied in the manner of a typical diaper inasmuch as the back side panels can be brought forward to engage the front side panels at a side seam to accommodate changes with the child in a supine position. Because the third disposable diaper configuration 60 can be applied in a prefastened configuration, stretch must be added to the diaper to accommodate being pulled over the hips while fastened. This stretch is preferably added in the form of stretch side panels 64 that also carry the fastening system 66 such that the third disposable diaper configuration 60 is fastened on the sides rather than in the front as is typical with a standard diaper. One or more side panels 64 can be attached refastenably to one another or to the chassis 62 to form a refastenable side seam 68. In other aspects, the one or more side panels 64 can be affixed to one another or to the chassis 62 to form a non-refastenable side seam (not shown). Such a side seam can be configured to be tearable.

The third disposable diaper configuration 60 can also include one or more tabs 70 attached to one or more side panels 64 to act as a disposal tab to secure the diaper for disposal and/or as a finger tab to increase the ease with which the refastenable side can be opened. The third disposable diaper configuration 60 is better able to accommodate the third level of cooperation because the prefastened configuration provides automatic fit due in large part to its stretchier configuration, and because it provides automatic centering through the ability to pull the diaper onto the wearer. Fit and centering are important to product performance, but can be otherwise difficult to achieve trying to apply a standard diaper to a squirmy child. Because the third disposable diaper configuration 60 is designed primarily for children who are squirmy or otherwise active during diaper changes, the third disposable diaper configuration 60 is structurally different from the second disposable diaper configuration.

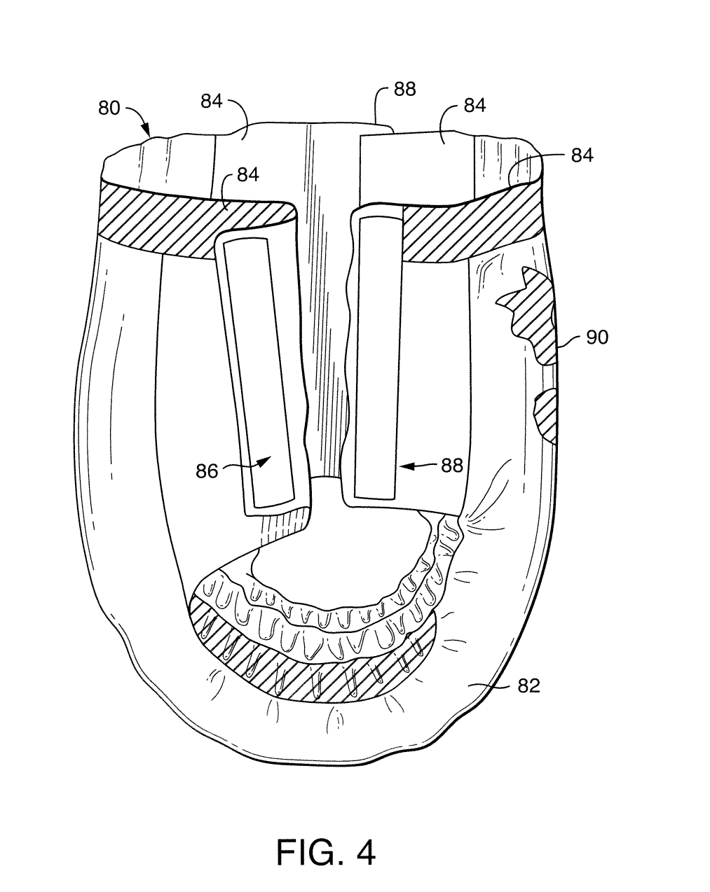

A fourth level of cooperation can cover a locomotive, toilet training child who might be capable of assisting in a change, and can be changed whether the child is cooperative or resistant. The array includes a fourth disposable article configuration 80 intended for use in conjunction with the fourth level of cooperation. An example of a fourth disposable diaper configuration 80 is illustrated in FIG. 4. The fourth disposable article configuration 80 includes a chassis 82 designed with specific features to correspond to a wearer's increased engagement and potential readiness for toilet training. The fourth disposable article configuration 80 has generally a pre-fastened configuration that allows a caregiver to pull the product onto a child. The fourth disposable article configuration 80 can also be unfastened by the caregiver and applied in the manner of a typical diaper inasmuch as the back side panels can be brought forward to engage the front side panels at a side seam to accommodate changes with the child in a supine position. Because the fourth disposable article configuration 80 can be applied in a prefastened configuration, stretch must be added to the article to accommodate being pulled over the hips while fastened. This stretch is preferably added in the form of stretch side panels 84 that also carry the fastening system 86 such that the fourth disposable article configuration 80 is fastened on the sides rather than in the front as is typical with a standard diaper. One or more side panels 84 can be attached refastenably to one another or to the chassis 82 to form a refastenable side seam 88. In other aspects, the one or more side panels 84 can be affixed to one another or to the chassis 82 to form a non-refastenable side seam (not shown). Such a side seam can be configured to be tearable.

The fourth disposable article configuration 80 can also include engaging and training-related features such as gender-specific graphics (not shown), active fading or appearing graphics 90, cooling material (not shown), wet-feeling material (not shown), or accommodation for any suitable wetness sensing or training aids. The fourth disposable article configuration 80 is generally configured to motivate cooperation, whereas the third disposable diaper configuration 60 is generally configured to accommodate non-cooperation.

In the parallel paths, the third disposable diaper configuration 60 can have a third configuration fastening means 72, and the second disposable diaper configuration 40 can have a second configuration fastening means 52, wherein the third configuration fastening means 72 is different from the second configuration fastening means 52. For example, the second configuration fastening means 52 can include ears 44 and fastening tabs 46, whereas the third configuration fastening means 72 can include side panels 64 and a refastenable seam 68. In another example, the third configuration fastening means can include a stretch configuration without side panels and a bonded and optionally tearable side seam. In other aspects, the second and third configuration fastening means 52, 72 can be any suitable fastening means, including those known to one skilled in the art.

To accomplish the parallel paths, the first disposable diaper configuration 20 can have a first size, and the second and third disposable diaper configurations 40, 60 can have a second size, wherein the second size is larger than the first size. In addition, each disposable diaper configuration can have more than one size. The sizes in which the first disposable diaper configuration 20 is available are preferably smaller than the sizes in which the second and third disposable diaper configurations 40, 60 are available, and the sizes in which the second disposable diaper configuration 40 is available are preferably similar or identical to the sizes in which the third disposable diaper configuration 60 is available. The sizes in which the fourth disposable article configuration 80 is available are preferably larger than the sizes in which the second and third disposable diaper configurations 40, 60 are available.

For instance, the first disposable diaper configuration 20 is available in first and second sizes, where the second size is larger than the first size. Both the second and third disposable diaper configurations 40, 60 are available in third and fourth sizes, where the third size is larger than the second size, and the fourth size is larger than the third size. The fourth disposable article configuration 80 is then available in fifth and sixth sizes, where the fifth size is bigger than the fourth size, and the sixth size is bigger than the fifth size. Because infants and toddlers experience levels of cooperation at different rates, multiple sizes can be provided for each disposable diaper configuration. As a result, different disposable diaper configurations can be made available in overlapping size ranges.

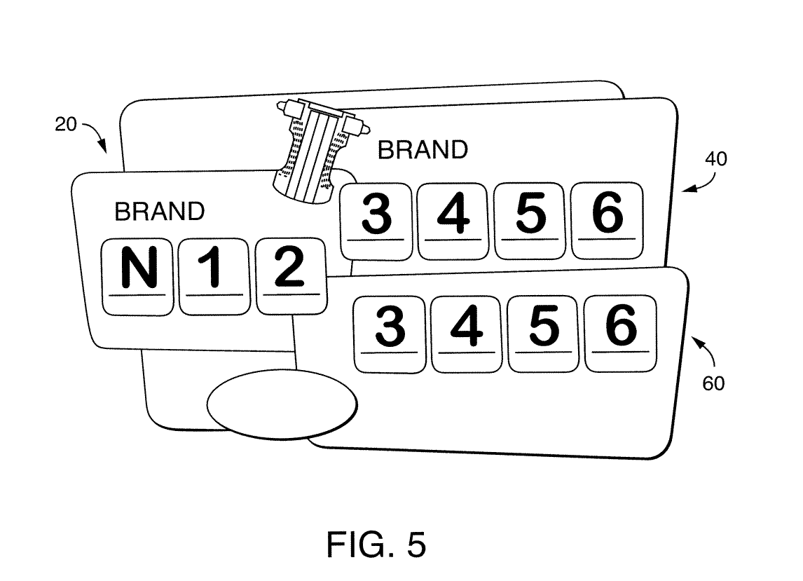

A specific example is illustrated in FIG. 5. A first disposable diaper configuration 20 labeled Little Snugglers is available in sizes N, 1, and 2, with weight ranges of up to 10 lbs., up to 14 lbs., and 12-18 lbs., respectively. In this example, the first disposable diaper configuration 20 can be available in size 3 as well with a weight range of 16-28 lbs. (not shown). Both a second disposable diaper configuration 40 labeled Little Movers and a third disposable diaper configuration 60 labeled Little Movers Slip-On Diapers are available in sizes 3, 4, 5, and 6, with weight ranges of 16-28 lbs., 22-37 lbs., over 27 lbs., and over 35 lbs., respectively. A fourth disposable article configuration (not shown) is available in sizes 2T-3T, 3T-4T, and 4T-5T with weight ranges 18-34 lbs., 32-40 lbs., and 38-50 lbs., respectively.

In a more general version of this example, a first disposable diaper configuration 20, including a chassis 22 designed with specific features to correspond to the first level of cooperation, includes a single progression of sizes. Second and third disposable diaper configurations 40, 60 each include a chassis 42, 62 designed with specific features to correspond to the second and third levels of cooperation, respectively. In this aspect, the third disposable diaper configuration 60 is structurally different from the second disposable diaper configuration 40, and the second and third disposable diaper configurations 40, 60 include parallel and identical progressions of sizes. A fourth disposable diaper configuration 80, including a chassis 82 designed with specific features to correspond to the fourth level of cooperation, includes another single progression of sizes.

Overlapping size ranges along parallel paths can make it difficult for a consumer to choose the right product configuration matching a particular wearers level of cooperation. For this reason, a merchandising system is provided that facilitates consumers' selection of a diaper from a variety of configurations. The system preferably includes indicia exhibiting pictorial representations of the disposable diaper configurations fitted to wearers at corresponding levels of cooperation. The indicia for the first disposable diaper configuration 20 might display an infant being held or asleep in a crib. Similarly, the indicia for the second disposable diaper configuration 40 might exhibit a baby awake and moving, while the indicia for the third disposable diaper configuration 60 might exhibit a mother changing the diaper while the wearer is standing.

By matching the level of cooperation of a wearer with the level of cooperation exhibited by the indicia, the consumer can choose the right product configuration for their particular wearer. The indicia can be provided on a display panel disposed above the store shelves on which the disposable diaper configurations are displayed for sale. Alternately, the indicia can be disposed on packaging for the different disposable diaper configurations or in advertisements disseminated to the public.

The merchandising system can also include different product names associating each product configuration with the particular level of cooperation for which the product is designed. For instance, the first disposable diaper configuration 20 can be named Little Snugglers, while the second and third product configurations 40, 60 can be named Little Movers and Little Movers Slip-On Diapers, respectively. Each of the product names listed are trade names of products manufactured by Kimberly-Clark Corporation.

There are several executional elements that help a consumer understand that the concepts described herein are options for diapers. Consumers have an option to select pull-on or slip-on diapers in addition to regular tab diapers based on the level of cooperation of their child. Having this option is more useful than having a single sequence of products that is traversed from tab-diapers to the start of potty training. These elements help consumers understand the role of this product form in the category and know that it is a new form of a diaper that is for the age range when babies start to wiggle, roll, and/or become difficult to change because they are more active.

Key elements that help a consumer know where these products fit in the panorama of offerings for a brand include:
Sub branding that includes or indicates a link to the age/stage of activity level of the baby. For example, for a HUGGIES brand diaper the link can be conveyed with a sub brand such as LITTLE MOVERS diapers such that consumers know this product is not for new babies. The sub brand should also indicate what type of diaper form the product is, such as a slip-on diaper or a pull-on diaper. To continue the example, HUGGIES LITTLE MOVERS Slip-On Diapers better conveys both the means of applying the product form and the age/stage for the target user, as opposed to previous examples wherein the sub-brand only conveys the means of applying the product form. Linking to the age/stage of activity level helps establish the product as a need that babies have generally at 4+ months rather than a sequential step for toddlers or potty training.
Communication to a consumer that a product is for babies who are active during changes can include textual clues such as “put on lying or standing” and/or “put on in any position.” Non-textual clues can include icons that show application by slipping on while lying down or stepping into the product, and using a picture of a baby who looks about 6-9 months old and looks as if they are about to get away (e.g., a mischievous look on a baby, a baby crawling away with just a diaper on because he/she didn't stay to get clothes on).
Communication to a consumer can also include wording or icons that convey the ease with which the product can be opened at the sides for removal and/or disposal.
Package and product aesthetics that convey a similarity in brand and application.

These and other modifications and variations to the present invention can be practiced by those of ordinary skill in the art, without departing from the spirit and scope of the present invention, which is more particularly set forth in the appended claims. In addition, it should be understood that aspects of the various aspects of the present invention can be interchanged either in whole or in part. Furthermore, those of ordinary skill in the art will appreciate that the foregoing description is by way of example only, and is not intended to limit the invention so further described in such appended claims.

What is claimed:

1. An array of disposable absorbent article configurations designed for daytime use by wearers at different levels of cooperation, the array comprising:
a first disposable diaper configuration including features configured to accommodate a first level of cooperation, wherein the first disposable diaper configuration is available in a first plurality of sizes;
a second disposable diaper configuration including features configured to accommodate a second level of cooperation, the second level of cooperation being different from the first level of cooperation, wherein the second disposable diaper configuration is available in a second plurality of sizes; and
a third disposable diaper configuration including features configured to accommodate a third level of cooperation, the third level of cooperation being different from the second level of cooperation, wherein the third disposable diaper configuration is structurally different from the second disposable diaper configuration, and wherein the third disposable diaper configuration is available in the second plurality of sizes.

2. The array of claim 1, wherein the absorbency of the second disposable diaper configuration at a given size is substantially the same as the absorbency of the third disposable diaper configuration at the given size.

3. The array of claim 1, wherein at least one of the second plurality of sizes is larger than each of the first plurality of sizes.

4. The array of claim 1, wherein each of the second plurality of sizes is larger than all of the first plurality of sizes.

5. The array of claim 1, further comprising a fourth disposable article configuration including features configured to accommodate a fourth level of cooperation, the fourth level of cooperation being different from the third level of cooperation, wherein the fourth disposable diaper configuration is structurally different from the third disposable diaper configuration, and wherein the fourth disposable diaper configuration is available in a third plurality of sizes.

6. The array of claim 5, wherein at least one of the third plurality of sizes is larger than each of the second plurality of sizes.

7. The array of claim 5, wherein the absorbency of the fourth disposable diaper configuration at a given size is less than the absorbency of the third disposable diaper configuration at the given size.

8. The array of claim 1, wherein the first and second disposable diaper configurations each include an ear and a fastening tab.

9. The array of claim 1, wherein the third disposable diaper configuration is manufactured in a pre-fastened condition.

10. The array of claim 1, wherein the third disposable diaper configuration includes two adjacent side panels joined at a side seam.

11. The array of claim 1, wherein the third disposable diaper configuration includes two adjacent side panels joined at a side seam and a tab disposed adjacent the seam.

12. The array of claim 11, wherein the tab is a disposal tab.

13. The array of claim 1, wherein the first disposable diaper configuration includes a first size, wherein the second disposable diaper configuration includes a second size, and wherein the third disposable diaper configuration includes the second size, the second size being larger than the first size.

14. The array of claim 1, wherein the first disposable diaper configuration includes first and second sizes, the second size being larger than the first size, wherein the second disposable diaper configuration includes third and fourth sizes, and wherein the third disposable diaper configuration includes the third and fourth sizes, the third size being larger than the second size, and the fourth size being larger than the third size.

15. The array of claim 1, wherein third disposable diaper configuration includes a third configuration fastening means, wherein the second disposable diaper configuration includes a second configuration fastening means, and wherein the third configuration fastening means is different from the second configuration fastening means.

16. The array of claim 15, wherein the first disposable diaper configuration includes the second configuration fastening means.

17. An array of disposable absorbent article configurations designed for daytime use by wearers at different levels of cooperation, the array comprising:
a first disposable diaper configuration including features configured to accommodate a first level of cooperation, wherein the first disposable diaper configuration is available in a first plurality of sizes;
a second disposable diaper configuration including features configured to accommodate a second level of cooperation, the second level of cooperation being different from the first level of cooperation, wherein the second disposable diaper configuration is available in a second plurality of sizes, and wherein at least one of the second plurality of sizes is larger than each of the first plurality of sizes; and
a third disposable diaper configuration including features configured to accommodate a third level of cooperation, the third level of cooperation being different from the second level of cooperation, wherein the third disposable diaper configuration is structurally different from the second disposable diaper configuration, and wherein the second and third disposable diaper configurations include parallel progressions of sizes.

18. The array of claim 17, wherein the second and third disposable diaper configurations include identical and parallel progressions of sizes.

19. An array of disposable absorbent article configurations designed for daytime use by wearers at different levels of cooperation, the array comprising:
a first disposable diaper configuration including features configured to accommodate a first level of cooperation;
a second disposable diaper configuration including features configured to accommodate a second level of cooperation, the second level of cooperation being different from the first level of cooperation, wherein the second disposable diaper configuration is available in a second plurality of sizes, and wherein the first and second disposable diaper configurations each have an ear and a fastening tab; and
a third disposable diaper configuration including features configured to accommodate a third level of cooperation, the third level of cooperation being different from the second level of cooperation, wherein the third disposable diaper configuration is available in the second plurality of sizes, and wherein the third disposable diaper configuration includes a side panel and a pre-fastened, refastenable side seam.

20. The array of claim 19, wherein the first disposable diaper configuration is available in a first plurality of sizes, and wherein at least one of the second plurality of sizes is larger than each of the first plurality of sizes.
